(19) United States
(12) Patent Application Publication
(10) Pub. No.: US 2024/0277310 A1
(43) Pub. Date: Aug. 22, 2024

(54) BLADDER VOLUME MEASURING DEVICE

(71) Applicant: EDGECARE INC., Seoul (KR)

(72) Inventors: Sang Eun PARK, Seoul (KR); Sang Whui WON, Hanam-si (KR); Hyun Woo CHO, Goyang-si (KR); Jung Jun KIM, Seoul (KR)

(73) Assignee: EDGECARE INC., Seoul (KR)

(21) Appl. No.: 18/436,792

(22) Filed: Feb. 8, 2024

(30) Foreign Application Priority Data
Feb. 16, 2023 (KR) 10-2023-0020573

(51) Int. Cl. A61B 8/08

(52) U.S. Cl. CPC A61B 8/08; A61B 8/5223

(57) ABSTRACT

According to the bladder volume measuring device according to the present invention, it is possible to calculate the shape coefficient based on information about the bladder shape generated from the determination unit that performs artificial intelligence learning on bladder ultrasound image big data and more accurately measure the amount of urine contained in the bladder by calculating the bladder volume based on the ultrasound image and the shape coefficient.

### CROSS REFERENCE TO RELATED APPLICATION

This application claims priority to the Korean Patent Application No. 10-2023-0020573 filed Feb. 16, 2023, the contents of which are incorporated herein by reference in its entirety.

### FIELD OF THE DISCLOSURE

The present invention relates to a bladder volume measuring device.

### BACKGROUND

When urine is sufficiently full in a bladder, a normal person may feel that the bladder is filled with enough urine to urinate, and excrete the urine to the outside. However, the number of patients who have difficulty excreting urine because the patients do not feel it even though the urine is sufficiently full in their bladder has been increasing recently. Various researches are currently being conducted to solve this problem.

### SUMMARY

The present invention provides a bladder volume measuring device capable of calculating a shape coefficient based on information about a bladder shape generated from a determination unit that performs artificial intelligence learning on bladder ultrasound image big data and more accurately measuring the amount of urine contained in the bladder by calculating the bladder volume based on the ultrasound image and the shape coefficient.

To solve the above problem, a bladder volume measuring device according to an embodiment of the present invention may include a first providing unit, a determination unit, a counting unit, and a calculation unit. The first providing unit may provide a first ultrasound image for a first cross section of a bladder. The determination unit may provide first shape information corresponding to information about a bladder shape based on the first ultrasound image. The counting unit may provide a corresponding shape coefficient according to the first shape information. The calculation unit may calculate a bladder volume according to the first ultrasound image and the shape coefficient.

The bladder volume measuring device may further include a second providing unit. The second providing unit may provide a second ultrasound image for a second cross section corresponding to a cross section perpendicular to the first cross section of the bladder.

The determination unit may provide shape information corresponding to the bladder shape based on the first ultrasound image and the second ultrasound image, the counting unit may provide a corresponding shape coefficient according to the shape information, and the calculation unit may calculate the bladder volume according to the first ultrasound image, the second ultrasound image, and the shape coefficient.

The counting unit may further include a score unit and a first counting auxiliary unit. The score unit may calculate a similarity score corresponding to a degree of similarity to each of the bladder shapes based on the shape information. The first counting auxiliary unit may calculate the shape coefficient according to the similarity score.

The counting unit may further include a reference unit. The reference unit may provide a reference score corresponding to the reference of the similarity score for calculating the shape coefficient.

The determination unit may further include an auxiliary determination unit. The auxiliary determination unit may provide shape auxiliary information corresponding to the bladder shape according to a third ultrasound image for a third cross section corresponding to a cross section that divides an angle formed by the first cross section and the second cross section in half.

The counting unit may further include an auxiliary score unit, a weighting unit, a calculation unit, and a second counting auxiliary unit. The auxiliary score unit may calculate an auxiliary score corresponding to the degree of similarity to each of the bladder shapes according to the shape auxiliary information. The weighting unit may determine a weight applied to the similarity score and the auxiliary score; The calculation unit may calculate a first weighted score and a second weighted score by applying the weight to the similarity score and the auxiliary score. The second counting auxiliary unit may calculate the shape coefficient according to the first weighted score and the second weighted score.

The determination unit may perform artificial intelligence learning on a plurality of ultrasound images and provide the first shape information corresponding to the first ultrasound image.

The calculation unit may further include a measuring unit. The measuring unit may measure width information, height information, and length information of the bladder from the first ultrasound image and the second ultrasound image.

The bladder volume measuring device may further include a notification unit. The notification unit may provide a notification signal when the bladder volume is greater than or equal to a predetermined reference volume.

In addition to the technical problems of the present invention described above, other features and advantages of the present invention will be described below, or may be clearly understood by those skilled in the art from such description and explanation.

### BRIEF DESCRIPTION OF DRAWINGS

FIG. 1 is a diagram illustrating a bladder volume measuring device according to an embodiment of the present invention.

FIGS. 2 to 4 are diagrams for describing the bladder volume measuring device of FIG. 1.

FIG. 5 is a diagram illustrating an embodiment of the bladder volume measuring device of FIG. 1.

FIG. 6 is a diagram for describing the bladder volume measuring device of FIG. 4.

FIG. 7 is a diagram illustrating a counting unit included in the bladder volume measuring device of FIG. 1.

FIG. 8 is a diagram for describing the counting unit included in the bladder volume measuring device of FIG. 1.

FIG. 9 is a diagram illustrating a determination unit and the counting unit included in the bladder volume measuring device of FIG. 1.

FIGS. 10 and 11 are diagrams for describing the determination unit and the counting unit included in the bladder volume measuring device of FIG. 1.

FIG. 12 is a diagram illustrating the determination unit included in the bladder volume measuring device of FIG. 1.

FIG. 13 is a diagram illustrating a calculation unit included in the bladder volume measuring device of FIG. 1.

FIG. 14 is a diagram illustrating a notification unit included in the bladder volume measuring device of FIG. 1.

### DETAILED DESCRIPTION

In this specification, in adding reference numerals to components throughout the drawings, it is to be noted that like reference numerals designate like components even though components are illustrated in different drawings.

On the other hand, the meaning of the terms described in the present specification should be understood as follows.

Singular expressions should be understood as including plural expressions, unless the context clearly defines otherwise, and the scope of rights should not be limited by these terms.

It should be understood that terms such as “include” and “have” do not preclude the existence or addition possibility of one or more other features or numbers, steps, operations, components, parts, or combinations thereof.

Hereinafter, preferred embodiments of the present invention designed to solve the above problems will be described in detail with reference to the accompanying drawings.

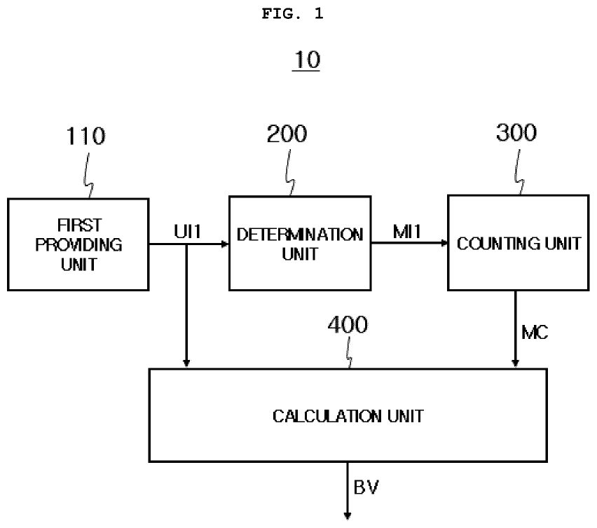

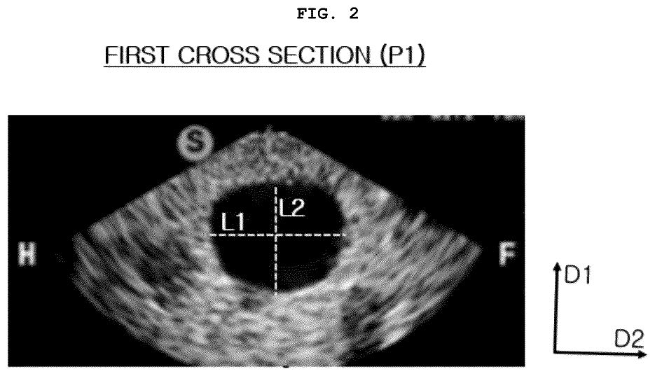

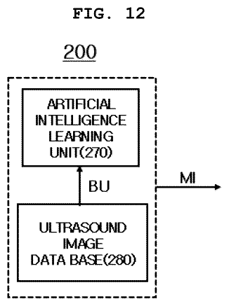

FIG. 1 is a diagram illustrating a bladder volume measuring device according to an embodiment of the present invention, FIGS. 2 to 4 are diagrams for describing the bladder volume measuring device of FIG. 1, and FIG. 12 is a diagram illustrating a determination unit included in the bladder volume measuring device of FIG. 1.

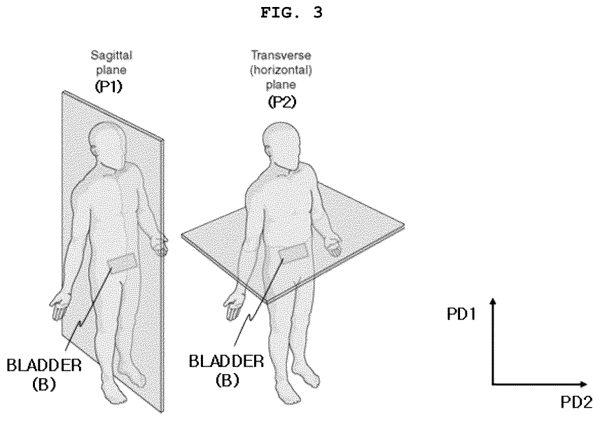

Referring to FIGS. 1 to 4 and 12, a bladder volume measuring device 10 according to an embodiment of the present invention may include a first providing unit 110, a determination unit 200, a counting unit 300, and a calculation unit 400. The first providing unit 110 may provide a first ultrasound image UI1 of a first cross section P1 of a bladder. For example, a cross section of a bladder placed inside a human body may be imaged using an ultrasound device. The first cross section P1 of the bladder may be included in a sagittal plane, and as illustrated in FIG. 3, the sagittal plane may be a cross section formed along a first plane direction PD1 in the human body. In addition, a second cross section P2 of the bladder may be included in a transverse plane, and as illustrated in FIG. 3, the transverse plane may be a cross section formed along a second plane direction PD2 in the human body. An angle formed by the first cross section P1 and the second cross section P2 may be 90°.

The determination unit 200 may provide first shape information MI1 corresponding to information about the bladder shape based on the first ultrasound image UI1. The bladder shape may vary from person to person. For example, the bladder shape may be divided into an ellipse, a rectangular parallelepiped, a triangular prism, and a sphere. The determination unit 200 according to the present invention may learn bladder ultrasound images BU included in an ultrasound image database 280 through artificial intelligence learning unit 270. When the determination unit 200, which has learned the big data provided from the ultrasound image database 280, receives the first ultrasound image UI1 for the first cross section P1 from the first providing unit 110, information about the bladder shape included in the first ultrasound image UI1 may be determined according to the artificial intelligence algorithm, and one of the ellipse, the rectangular parallelepiped, the triangular prism, and the sphere may be selected and provided as the first shape information MI1.

The counting unit 300 may provide a corresponding shape coefficient MC according to the first shape information MI1. When the first shape information MI1 is transmitted from the determination unit 200, the counting unit 300 may provide the shape coefficient MC corresponding to the first shape information MI1. For example, when the first shape information MI1 is an ellipse, the shape coefficient MC corresponding to the ellipse may be 0.81, and when the first shape information MI1 is the rectangular parallelepiped, the shape coefficient MC corresponding to the rectangular parallelepiped may be 0.89. In the same way, the shape coefficient MC for the triangular prism may be 0.66, and the shape coefficient MC for the sphere may be 0.52. The shape coefficient MC may be used in the calculation unit 400 to calculate a bladder volume BV.

The calculation unit 400 may calculate the bladder volume BV according to the first ultrasound image UI1 and the shape coefficient MC. In one embodiment, the calculation unit 400 may calculate the bladder volume BV according to width information W, height information H, and the shape coefficient MC of the bladder calculated from the first ultrasound image UI1. For example, the width of the bladder corresponding to the width information W of the bladder may be a first length L1, and a height corresponding to the height information H of the bladder may be a second length L2. In addition, the first shape information MI1 provided from the determination unit 200 may be spherical, and the shape coefficient MC corresponding to the sphere may be 0.52. In this case, the calculation unit 400 may calculate the bladder volume BV based on the first length L1, the second length L2, and 0.52.

According to the bladder volume measuring device 10 according to the present invention, it is possible to calculate the shape coefficient based on a shape information MI of the bladder generated from the determination unit 200 that performs the artificial intelligence learning on the bladder ultrasound image big data and more accurately measure the amount of urine contained in the bladder by calculating the bladder volume BV based on the ultrasound image and the shape coefficient MC.

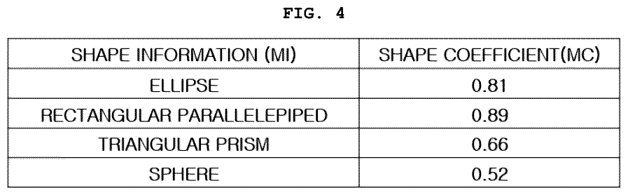

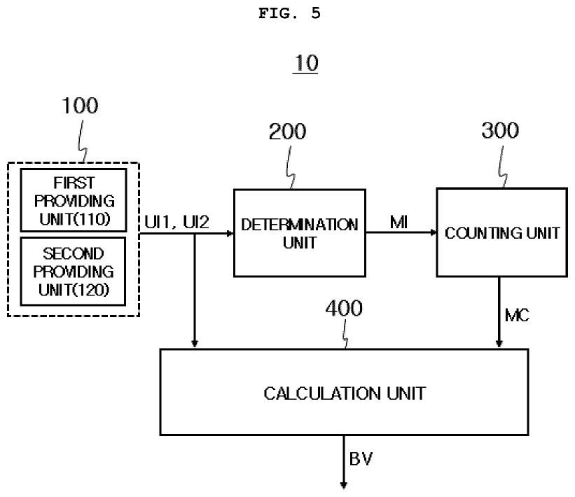

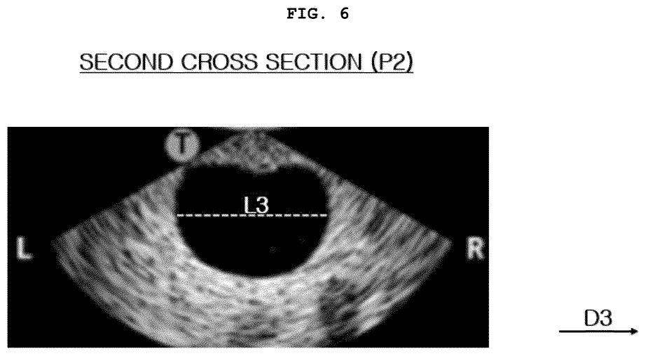

FIG. 5 is a diagram illustrating an embodiment of the bladder volume measuring device of FIG. 1 and FIG. 6 is a diagram for describing the bladder volume measuring device of FIG. 4.

Referring to FIGS. 1 to 6, in one embodiment, the bladder volume measuring device 10 may further include a second providing unit 120. The second providing unit 120 may provide a second ultrasound image UI2 for the second section P2 corresponding to a cross section perpendicular to the first section P1 of the bladder.

The determination unit 200 may provide the shape information MI corresponding to the bladder shape based on the first ultrasound image UI1 and the second ultrasound image UI2. For example, the determination unit 200 according to the present invention may learn a bladder ultrasound images BU included in the ultrasound image database 280 through the artificial intelligence learning. When the determination unit 200, which has learned the big data provided from the ultrasound image database 280, receives the first ultrasound image UI1 for the first cross section P1 and the second cross section P2 from the first providing unit 110 and the second providing unit 120, the information about the bladder shape included in the first ultrasound image UI1 and the second ultrasound image UI2 may be determined according to the artificial intelligence algorithm, and one of the ellipse, the rectangular parallelepiped, the triangular prism, and the sphere may be selected and provided as the shape information MI.

The counting unit 300 may provide the corresponding shape coefficient MC according to the shape information MI1. The calculation unit 400 may calculate the bladder volume BV according to the first ultrasound image UI1, the second ultrasound image UI2, and the shape coefficient MC. In one embodiment, the calculation unit 400 may calculate the bladder volume BV according to the width information W, the height information H, the length information L, and the shape coefficient MC of the bladder calculated from the first ultrasound image UI1 and the second ultrasound image UI2. For example, the width of the bladder corresponding to the width information W of the bladder may be the first length L1, the height corresponding to the height information H of the bladder may be the second length L2, the length corresponding to the length information L of the bladder may be a third length L3. In addition, the first shape information MI1 provided from the determination unit 200 may be elliptical, and the shape coefficient MC corresponding to the ellipse may be 0.81. In this case, the calculation unit 400 may calculate the bladder volume BV by multiplying the first length L1, the second length L2, the third length L3, and 0.81.

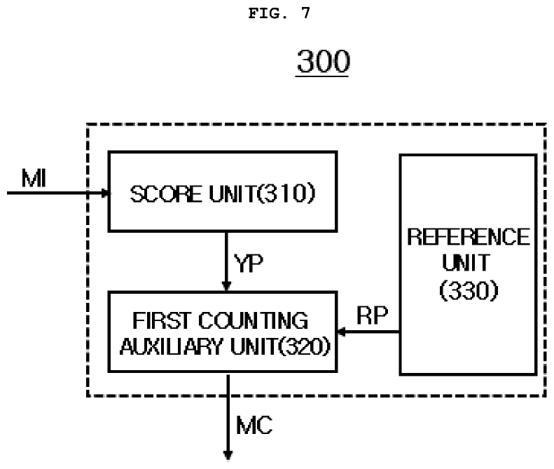

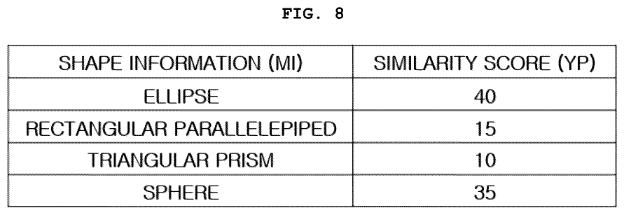

FIG. 7 is a diagram illustrating the counting unit included in the bladder volume measuring device of FIG. 1 and FIG. 8 is a diagram for describing the counting unit included in the bladder volume measuring device of FIG. 1.

Referring to FIGS. 1 to 8, in one embodiment, the counting unit 300 may further include a score unit 310 and a first counting auxiliary unit 320. The score unit 310 may calculate a similarity score YP corresponding to a degree of similarity to each of the bladder shapes based on the shape information MI. There may be cases where it is not clear whether the bladder shape is an ellipse or a sphere according to the shape information MI provided by the determination unit 200. In this case, the shape coefficient MC corresponding to the bladder shape may be determined using the similarity score YP. When a difference in similarity score YP is smaller than or equal to a predetermined difference score, the shape information MI may be considered not to be clearly distinguished. For example, according to the shape information MI provided from the determination unit 200 based on the first ultrasound image UI1 and the second ultrasound image UI2, a first similarity score YP corresponding to the degree of similarity to the ellipse may be 40 points, and a second similarity score YP corresponding to the degree of similarity to the rectangular parallelepiped may be 15 points. In addition, a third similarity score YP corresponding to the degree of similarity to the triangular prism may be 10 points, and a fourth similarity score YP corresponding to the degree of similarity to the sphere may be 15 points. Here, when the difference score is 7 points, the difference between the first similarity score YP of 40 points and the fourth similarity score YP of 35 points is 5 points, and therefore, may be smaller than the difference number of 7 points. In this case, the counting unit 300 may determine that the shape coefficient MC may not be determined according to the shape information MI, and the shape coefficient MC may be calculated using the first counting auxiliary unit 320 below.

The first counting auxiliary unit 320 may calculate the shape coefficient MC according to the similarity score YP. In one embodiment, the counting unit 300 may further include a reference unit 330. The reference unit 330 may provide a reference score RP corresponding to a reference of the similarity score YP for calculating the shape coefficient MC. For example, the reference score RP may be 30 points. In this case, the first counting auxiliary unit 320 may determine the shape coefficient MC according to the similarity score YP of the shape information MI, which is higher than the reference score RP. As illustrated in FIG. 8, the bladder shape having the reference score RP greater than or equal to 30 points may be elliptical or spherical. In this case, the first counting auxiliary unit 320 sums a first coefficient obtained by multiplying 0.81, which is the shape coefficient MC for the ellipse, by 40/(40+35), and a second coefficient obtained by multiplying 0.52, which is the shape coefficient MC for the sphere by 35/(40+35) to provide the shape coefficient MC.

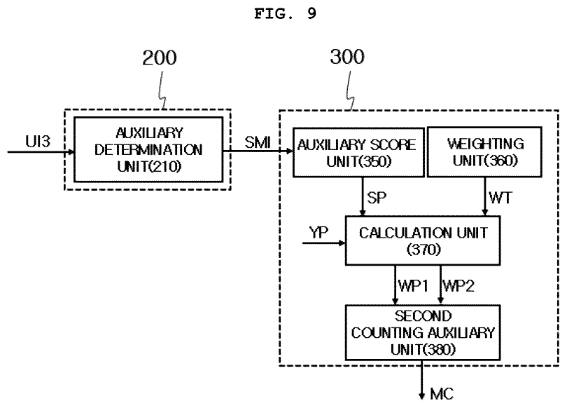

FIG. 9 is a diagram illustrating a determination unit and a counting unit included in the bladder volume measuring device of FIG. 1 and FIGS. 10 and 11 are diagrams for describing the determination unit and counting unit included in the bladder volume measuring device of FIG. 1.

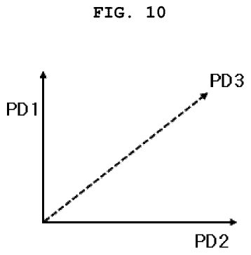

Referring to FIGS. 1 to 12, in one embodiment, the determination unit 200 may further include an auxiliary determination unit 210. The auxiliary determination unit 210 provides shape auxiliary information SMI corresponding to the bladder shape according to a third ultrasound image UI3 of a third cross section corresponding to the cross section that divides the angle formed by the first cross section P1 and the second cross section P2 in half. For example, there may be a case where the bladder shape may not be determined using the first ultrasound image UI1 for the first cross section P1 and the second ultrasound image UI1 for the second cross section P2. In this case, the bladder shape may be determined more accurately using the third cross section provided to the auxiliary determination unit 210. As illustrated in FIG. 10, the third cross section may be a cross section that divides the angle between the first cross section P1 and the second cross section P2 in half. The auxiliary determination unit 210 may provide the shape auxiliary information SMI that may auxiliary determine the bladder shape based on the third ultrasound image UI3 corresponding to the third cross section.

In one embodiment, the counting unit 300 may further include an auxiliary score unit 350, a weighting unit 360, a calculation unit 370, and a second counting auxiliary unit 380. The auxiliary score unit 350 may calculate an auxiliary score SP corresponding to the degree of similarity to each of the bladder shapes according to the shape auxiliary information SMI. For example, according to the shape auxiliary information SMI corresponding to the third ultrasound image UI3 provided from the determination unit 200, a first auxiliary score SP1 corresponding to the degree to which the bladder shape is similar to the ellipse may be 80 points, and a second auxiliary score SP2 corresponding to the degree of similarity to the sphere may be 20 points.

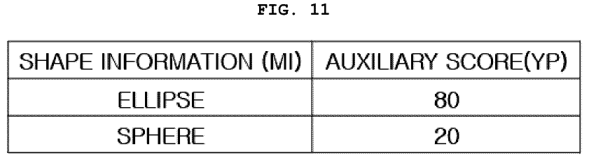

The weighting unit 360 may determine a weight WT applied to the similarity score YP and the auxiliary score SP. The calculation unit 370 may calculate a first weighted score WP1 and a second weighted score WP2 by applying the weight WT to the similarity score YP and the auxiliary score SP. For example, a first weight WT applied to the similarity score YP may be 1, and a second weight WT applied to the auxiliary score SP may be 0.5. Referring to FIGS. 8 and 11, the first weighted score WP1 may include a first weighted score_ellipse and a first weighted score_sphere, and the first weighted score_ellipse may be 40 points*1=40 points and the first weighted score_sphere may be 35 points*1=35 points. The second weighted score_WP2 may be include a second weighted score_ellipse and a second weighted score_sphere, and the second weighted score_ellipse may be 80 points*0.5=40 points, and the second weighted score_sphere may be 20*0.5=10 points.

The second counting auxiliary unit 380 may calculate the shape coefficient according to the first weighted score WP1 and the second weighted score WP2. For example, the shape coefficient MC for the ellipse may be 0.81, and the shape coefficient MC for the sphere may be 0.52. In addition, the sum of the first weighted score_ellipse and the second weighted score_ellipse may be 80 points, and the sum of the first weighted score_sphere and the second weighted score_sphere may be 45 points. In this case, the shape coefficient MC provided by the second counting auxiliary unit 380 may be 0.81*80/(80+45)+0.52*45/(80+45)=0.706 which is a weighted average value of the shape coefficient MC for the ellipse and sphere with the first weighted score WP1 and the second weighted score WP2.

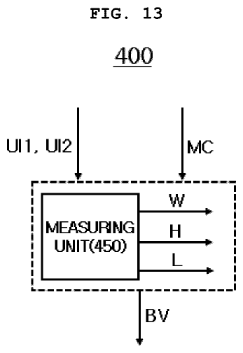

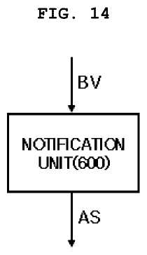

FIG. 13 is a diagram illustrating a calculation unit included in the bladder volume measuring device of FIG. 1 and FIG. 14 is a diagram illustrating a notification unit included in the bladder volume measuring device of FIG. 1.

Referring to FIGS. 1 to 14, in one embodiment, the calculation unit 400 may further include a measuring unit 450. The measuring unit 450 may measure the width information W, the height information H, and the length information L of the bladder from the first ultrasound image UI1 and the second ultrasound image UI2. For example, the width of the bladder corresponding to the width information of the bladder calculated based on the first ultrasound image UI1 and the second ultrasound image UI2 may be the first length L1, the height corresponding to the height information of the bladder may be the second length L2, and the length corresponding to the length information of the bladder may be the third length L3. In addition, the first shape information MI1 provided from the determination unit 200 may be elliptical, and the shape coefficient MC corresponding to the ellipse may be 0.81. In this case, the calculation unit 400 may calculate the bladder volume BV by multiplying the first length L1, the second length L2, the third length L3, and 0.81.

In one embodiment, the bladder volume measuring device 10 may further include a notification unit 600. The notification unit may provide a notification signal AS when the bladder volume BV is greater than or equal to a predetermined reference volume For example, when a patient's bladder is filled with urine more than a reference volume, the bladder volume measuring device 10 according to the present invention may request a patient to excrete urine by transmitting a notification signal to a caregiver's smartphone.

According to the bladder volume measuring device 10 according to the present invention, it is possible to calculate the shape coefficient based on the shape information MI of the bladder generated from the determination unit 200 that performs the artificial intelligence learning on the bladder ultrasound image big data and more accurately measure the amount of urine contained in the bladder by calculating the bladder volume BV based on the ultrasound image and the shape coefficient MC.

According to the present invention as described above, the following effects are obtained.

According to the bladder volume measuring device according to the present invention, it is possible to calculate the shape coefficient based on the information about the bladder shape generated from the determination unit that performs the artificial intelligence learning on the bladder ultrasound image big data and more accurately measure the amount of urine contained in the bladder by calculating the bladder volume based on the ultrasound image and the shape coefficient.

In addition, other features and advantages of the present invention may be newly understood through the embodiments of the present invention.

In addition to the technical problems of the present invention described above, other features and advantages of the present invention will be described below, or may be clearly understood by those skilled in the art from such description and explanation.

1. A bladder volume measuring device, comprising:
a first providing unit that provides a first ultrasound image for a first cross section of a bladder;
a determination unit that provides first shape information corresponding to information about a bladder shape based on the first ultrasound image;
a counting unit that provides a corresponding shape coefficient according to the first shape information; and
a calculation unit that calculates a bladder volume according to the first ultrasound image and the shape coefficient.

2. The bladder volume measuring device of claim 1, further comprising:
a second providing unit that provides a second ultrasound image for a second cross section corresponding to a cross section perpendicular to the first cross section of the bladder.

3. The bladder volume measuring device of claim 2, wherein the determination unit provides shape information corresponding to the bladder shape based on the first ultrasound image and the second ultrasound image,
the counting unit provides a corresponding shape coefficient according to the shape information, and
the calculation unit calculates the bladder volume according to the first ultrasound image, the second ultrasound image, and the shape coefficient.

4. The bladder volume measuring device of claim 3, wherein the counting unit includes:
a score unit that calculates a similarity score corresponding to a degree of similarity to each of the bladder shapes based on the shape information; and
a first counting auxiliary unit that calculates the shape coefficient according to the similarity score.

5. The bladder volume measuring device of claim 4, wherein the counting unit further includes a reference unit that provides a reference score corresponding to a reference of the similarity score for calculating the shape coefficient.

6. The bladder volume measuring device of claim 5, wherein the determination unit includes an auxiliary determination unit that provides shape auxiliary information corresponding to the bladder shape according to a third ultrasound image for a third cross section corresponding to a cross section that divides an angle formed by the first cross section and the second cross section in half.

7. The bladder volume measuring device of claim 6, wherein the counting unit further includes:
an auxiliary score unit that calculates an auxiliary score corresponding to the degree of similarity to each of the bladder shapes according to the shape auxiliary information;
a weighting unit that determines a weight applied to the similarity score and the auxiliary score;
a calculation unit that calculates a first weighted score and a second weighted score by applying the weight to the similarity score and the auxiliary score; and
a second counting auxiliary unit that calculates the shape coefficient according to the first weighted score and the second weighted score.

8. The bladder volume measuring device of claim 6, wherein the determination unit performs artificial intelligence learning on a plurality of ultrasound images and provides the first shape information corresponding to the first ultrasound image.

9. The bladder volume measuring device of claim 8, wherein the calculation unit includes a measuring unit that measures width information, height information, and length information of the bladder from the first ultrasound image and the second ultrasound image.

10. The bladder volume measuring device of claim 9, further comprising:
a notification unit that provides a notification signal when the bladder volume is greater than or equal to a predetermined reference volume.
